(19) United States
(12) Patent Application Publication
(10) Pub. No.: US 2006/0078569 A1
(43) Pub. Date: Apr. 13, 2006

(54) Method for recovery of photo-inhibited immunological activity

(76) Inventors: Hirotaka Kakita, Takamatsu-shi (JP); Hiroshi Kamishima, Takamatsu-shi (JP)

Correspondence Address: WENDEROTH, LIND & PONACK, L.L.P., 2033 K STREET N. W., SUITE 800, WASHINGTON, DC, 20006-1021, US

(21) Appl. No.: 11/241,926

(22) Filed: Oct. 4, 2005

(30) Foreign Application Priority Data
Oct. 4, 2004 (JP) 2004-318566

(51) Int. Cl. A61K 36/02

(52) U.S. Cl. 424/195.17

(57) ABSTRACT

The present invention provides a novel method for recovery of photo-inhibited immunocompetence, which comprises administration of a specific active ingredient exhibiting physiological activity such as activity of activating cellular immunocompetence, which is obtained from a raw material that exists in a large amount in the nature and can easily be acquired. The above active ingredient is obtained in the form of a liquid crude active fraction by: adding ammonium sulfate to an extract obtained from Gracilaria sp., using an aqueous salt solution, to a final concentration of 20% to 40% saturated solution to conduct a first stage of salting-out, so as to eliminate the precipitated contaminants; further adding ammonium sulfate to the extract to a final concentration of 60% to 80% saturated solution to conduct a second stage of salting-out, so as to recover a crude active fraction as a precipitate; and dissolving the precipitate in a suitable solvent. It is also obtained by dissolving the above precipitate in a buffer solution, allowing the obtained solution to come into contact with a dialyzing fluid that has been adjusted to the isoelectric point of a substance exhibiting physiological activity, via a dialysis membrane, so as to transfer low molecular weight impurities to the dialyzing fluid and eliminate them, and at the same time, precipitating a physiologically active polymer from a solution containing high molecular weight impurities and recovering it.

### BACKGROUND OF THE INVENTION

The present invention relates to a method for recovery of photo-inhibited immunocompetence, which recovers the immunocompetence of the skin that has been damaged by ultraviolet rays or other types of radiations, so as to recover a healthy skin and a method for producing an agent used for the above method, using Gracilaria sp. as a raw material.

One of the most important biophylaxis functions is the immune function of the skin. The skin is an organ existing on the outermost layer of a living body. Thus, it is most likely to suffer physical, chemical, or biological attack from the outside. In order to prevent such damage from penetrating into internal tissues, the skin has an immune function.

The skin is composed of: epidermal tissues consisting of keratinocytes and Langerhans cells; and dermal tissues consisting of dendritic cells, vascular endothelial cells, macrophages, and others. Among these cells, Langerhans cells rapidly respond to an antigen that enters from the outside and acts as foreign matter, and transmit the information to T cells via lymph nodes. Thus, it is known that Langerhans cells play an important role regarding a series of immunological reactions.

By the way, if the skin is exposed to radiations such as ultraviolet rays for a long period of time, cells are damaged, and the aforementioned immune functions are lost, resulting in a decrease in the defense function of the skin.

Accordingly, studies regarding activation of such lost immune function of the skin have been conducted. Several immunostimulators or immunoactivators have been proposed. Examples of such immunostimulators may include: an immunostimulator comprising, as an active ingredient, a substance extracted from an marine algae belonging to Gracilaria sp., using an aqueous solvent (refer to JP 5-139988A); an immunostimulator comprising, as an active ingredient, a solid, which is separated from a solution obtained by extracting acidic polysaccharide from seaweed belonging to red algae using an aqueous solvent, followed by solid-liquid separation, and then allowing β-agarase having ability to hydrolyze the above acidic polysaccharide to act on the obtained extract, so as to reduce the viscosity of the acidic polysaccharide, or a solution obtained by allowing the aforementioned seaweed to come into contact with an aqueous solvent containing P-agarase, and extracting acidic polysaccharide as well as decreasing the viscosity thereof, followed by solid-liquid separation, or a solid, which is separated from solution purified from these solutions (refer to JP 6-256208A); and an immunostimulator obtained by treating a whey protein concentrate by column chromatography, so as to obtain a glycomacropeptide-rich fraction, and then subjecting the obtained fraction to gel filtration and then to ion exchange chromatography (refer to JP 9-12474A).

When these immunostimulators are orally administered, they act to recover the decreased immune function of the skin. However, since the action mechanism of these agents regarding stimulation of the skin immunity, it is totally unknown regarding whether or not these agents have the same above effects when they are applied to external use.

Moreover, as a skin immunostimulator that can be applied as an external preparation, a skin immunostimulator containing a combination of glutathione with carotenes, xanthines, furans, tocopherols, or amino acids is proposed (refer to JP 11-292737A). However, it cannot be said that this immunostimulator is always satisfactory in respect of effectiveness.

Hemagglutinin exhibits a specific behavior to the erythrocytes of various animals. Hence, hemagglutinin has been widely used as a test reagent or a separating material in various fields such as medical, pharmaceutical, or biochemical field. Such hemagglutinin is broadly divided into that derived from animals and that derived from plants. Taking into consideration availability in a large volume or handlability, hemagglutinin derived from plants has become a focus of attention for practical use.

Examples of hemagglutinin derived from plants that is known to date may include: those derived from land plants, such as concanavalin A (Con A) from jack beans, or wheat germ lectin (WGA) from wheat (refer to J. Bacteriol., 1936, vol. 32, pp. 227-237); and those derived from marine plants, such as GVAI from Gracilaria verrucosa, or Hypnin A, B, C, and D from Hypnea japonica (refer to Bul. Jap. Soc. Sci. Fishe., 1981, vol. 47, pp. 1079-1084).

With regard to hemagglutinin derived from land plants, a sample with high hemagglutination activity can be relatively easily obtained. However, since such hemagglutination activity is inhibited even by simple sugar such as monosaccharide or disaccharide, the above hemagglutinin is disadvantageous in terms of low recognition carbohydrate selectivity. In the case of hemagglutinin derived from marine plants, the hemagglutination activity thereof is not inhibited by monosaccharide or disaccharide, but it is inhibited by glycoproteins such as fetuin or asialofetuin. Thus, hemagglutinin derived from marine plants is considered to have high recognition carbohydrate selectivity, but it is disadvantageous in that it is difficult to obtain a sample with high hemagglutination activity. Moreover, both the above hemagglutinins are disadvantageous in that they cannot control hemagglutination activity due to a change in ionic strength.

Furthermore, in general, hemagglutinin is disadvantageous in that it loses its carbohydrate binding ability as a result of a heat treatment at 100° C.

A breakthrough physiological activity of hemagglutinin on cells is a reaction thereof with lymphocytes. When lymphocytes are cultured together with hemagglutinin having an extremely low concentration, lymphocytes proliferate and then start to divide. Such an effect of inducing lymphocytes that are in a resting stage to a growth and proliferation stage is known as mitogenic stimulation. This mitogenic stimulation is an important phenomenon that can be a key to the immune reaction of a living body to foreign matter (antigen). Mitogenic stimulation function is one of cellular immunocompetence-stimulating functions. This can constitute an indicator of the natural immunity-enhancing activity of hemagglutinin.

Examples of hemagglutinin that is mainly used as a mitogen may include concanavalin A (Con A), red kidney bean (Phaseolus vulgaris) lectin P (PHA-P), red kidney bean (Phaseolus vulgaris) lectin L (PHA-L), and pokeweed lectin (PWM). Such hemagglutinin is tested by culturing together with lymphocytes for 48 to 72 hours and then by measuring the increasing rate of labeled thymidine incorporated into the DNA.

Hemagglutinin with mitogenic ability is able to activate almost all lymphocytes that can be activated, regardless of the antigen specificity of cells. Thus, using such hemagglutinin with mitogenic ability, the pursuit of a change in cell growth or the study thereof can easily be carried out. In addition, it has been clarified that such hemagglutinin induces T cells to exhibit cytotoxicity. Since the induced cytotoxicity of T cells is non-specific to antigens, it acts on various normal cells and malignant cells.

Thus, since mitogenic activation by hemagglutinin can easily and simply be used, it can be used as means for determining the immunocompetence of patients suffering from various diseases including AIDS. Moreover, it is also used for the purpose of examining various immunosuppressive effects or the effects of immunotherapy. Furthermore, such hemagglutinin has recently attracted attention also as a lymphocyte division promoter in LAK therapy that is a new therapy for cancers.

Hemagglutinin has ability to specifically recognize a carbohydrate and bind thereto. With this characteristic, when hemagglutinin is directly administered into a living body or percutaneously administered to the skin as a mitogen, it recognizes the carbohydrate of a cell cortex and binds to the cell. Thus, when compared with a mitogen with no carbohydrate binding ability, the hemagglutinin binds to the carbohydrate of a cell cortex, so as to approach to the cell cortex, and it can thereby exhibit the function as a mitogen more effectively.

Nevertheless, such hemagglutinin with mitogenic ability comprises a protein as a main component, and thus it loses such carbohydrate binding ability, when it is subjected to a heat treatment at a high temperature (approximately 100° C.) or when it is maintained at a temperature between 40° C. and 50° C. for a long period of time. Accordingly, the combined use of hemagglutinin with other reagents when such hemagglutinin is administered into a living body, or the combined use thereof with other components when it is administered to the skin as cream or ointment, should be limited. Under such circumstances, it has been desired that hemagglutinin with mitogenic ability, which maintains carbohydrate binding ability even after being subjected to a heat treatment, be developed. The present inventors had previously proposed a method for producing highly active hemagglutinin from Gracilaria sp. (JP 7-278004A). However, it had not been known that such highly active hemagglutinin has physiological activity such as action to recover photo-inhibited immunocompetence.

### SUMMARY OF THE INVENTION

It is an object of the present invention to provide a novel agent for recovery of photo-inhibited immunocompetence, which is produced from a raw material that exists in large amount in the nature and that is easily acquired, and which has strong action to recover photo-inhibited immunocompetence and can be used via an oral or parenteral administration route.

The term “agent for recovery of photo-inhibited immunocompetence” is used herein to mean a substance having ability to recover the immune functions of a skin organ that are damaged by irradiation of ultraviolet rays or other types of radiations and to regenerate an organ having the original immunocompetence.

As a result of various studies regarding the physiological activity of a biopolymeric substance derived from marine algae, and in particular, hemagglutinin, the present inventors have found that a liquid extract obtained from Gracilaria sp. using an aqueous salt solution activates cellular immunocompetence, and that it is useful as an agent for recovery of photo-inhibited immunocompetence. Based on these findings, the inventors have completed the present invention.

That is to say, the present invention provides a method for recovery of photo-inhibited immunocompetence, using an agent containing, as an active ingredient, a liquid extract obtained from Gracilaria sp. using an aqueous salt solution.

This agent for recovery of photo-inhibited immunocompetence is produced by the following methods (1) to (4), for example.

(1) a method, which comprises: obtaining an extract from Gracilaria sp., using an aqueous salt solution; first adding ammonium sulfate to the obtained extract to a final concentration of 20% to 40% saturated solution to conduct a first stage of salting-out, so as to eliminate the precipitated contaminants; further adding ammonium sulfate to the extract to a final concentration of 60% to 80% saturated solution to conduct a second stage of salting-out, so as to recover a crude active fraction exhibiting activity of activating cellular immunocompetence; and in some cases, dissolving the recovered precipitate in a solvent, so as to prepare a solution,

(2) a method, which comprises: obtaining an extract from Gracilaria sp., using an aqueous salt solution; first adding ammonium sulfate to the obtained extract to a final concentration of 20% to 40% saturated solution to conduct a first stage of salting-out, so as to eliminate the precipitated contaminants; further adding ammonium sulfate to the extract to a final concentration of 60% to 80% saturated solution to conduct a second stage of salting-out, so as to separate the generated precipitate; dissolving the precipitate in a solvent, so as to prepare a solution; and subjecting the above described solution to a heat treatment at a temperature of 100° C. for 10 minutes, so as to eliminate contaminants in the form of precipitates, so as to obtain an active fraction exhibiting activity of activating cellular immunocompetence,

(3) a method, which comprises: obtaining an extract from Gracilaria sp., using an aqueous salt solution; first adding ammonium sulfate to the obtained extract to a final concentration of 20% to 40% saturated solution to conduct a first stage of salting-out, so as to eliminate the precipitated contaminants; further adding ammonium sulfate to the extract to a final concentration of 60% to 80% saturated solution to conduct a second stage of salting-out, so as to separate the generated precipitate; dissolving the above described precipitate in a buffer solution, so as to prepare a solution containing a substance exhibiting activity of activating cellular immunocompetence and impurities; allowing this solution to come into contact with a dialyzing fluid that has been adjusted to the isoelectric point of the substance exhibiting activity of activating cellular immunocompetence, via a dialysis membrane, so as to transfer low molecular weight impurities to the dialyzing fluid and eliminate them, and at the same time, precipitating a physiologically active polymer from a solution containing high molecular weight impurities and recovering it, and

(4) a method, which comprises: obtaining an extract from Gracilaria sp., using an aqueous salt solution; first adding ammonium sulfate to the obtained extract to a final concentration of 20% to 40% saturated solution to conduct a first stage of salting-out, so as to eliminate the precipitated contaminants; further adding ammonium sulfate to the extract to a final concentration of 60% to 80% saturated solution to conduct a second stage of salting-out, so as to separate the generated precipitate; dissolving the above described precipitate in a buffer solution, so as to prepare a solution containing a substance exhibiting activity of activating cellular immunocompetence and impurities; allowing this solution to come into contact with a dialyzing fluid that has been adjusted to the isoelectric point of high molecular weight impurities, via a dialysis membrane, so as to transfer low molecular weight impurities to the dialyzing fluid and eliminate them, and at the same time, precipitating the high molecular weight impurities and separating them, so as to obtain a solution exhibiting activity of activating cellular immunocompetence. As the aforementioned dialysis membrane, a regenerated cellulose tube is preferable.

The thus produced agent that is used in the method for recovery of photo-inhibited immunocompetence has any one of the following characteristics (i) to (x):

(i) Gracilaria sp. is characterized in that neither male gametophytes nor female gametophytes are detectable as mature bodies or thalluses in the nature and only tetrasporophytes are detectable as mature bodies, wherein the Gracilaria sp. grows in a natural seawater area, into which fresh water is mixed,

(ii) Gracilaria sp. is an immature unialgal culture strain derived from Gracilaria sp., which is characterized in that neither male gametophytes nor female gametophytes are detectable as mature bodies in the nature and only tetrasporophytes are detectable as mature bodies, and which grows in a natural seawater area, into which fresh water is mixed, or an algal body by proliferation of the immature unialgal culture strain (the term “immature” means “a property of rarely growing up to maturity”),

(iii) the liquid extract has a property to agglutinate sheep erythrocytes treated with pronase, and the agglutination activity is not inhibited by monosaccharide or disaccharide, but is inhibited by fetuin or asialofetuin,

(iv) the agglutination activity of the liquid extract on rabbit erythrocytes is changed according to ionic strength,

(v) the liquid extract has blast transformation activity on human lymphocytes,

(vi) the liquid extract promotes incorporation of tritium-labeled thymidine into the cell nucleus,

(vii) the liquid extract has carbohydrate binding activity even after it has been subjected to a heat treatment at 100° C. for 10 minutes,

(viii) the liquid extract comprises sugar and protein, and the ratio of the mass of the above described protein to that of the sugar is 0.4 or less,

(ix) the liquid extract comprises 1% to 60% by mass of sulfuric acid, and

(x) the liquid extract comprises a component eluted in a fraction that corresponds to a molecular weight of 100,000 or more in gel filtration chromatography using a globular protein as a standard molecular weight substance.

### DETAILED DESCRIPTION OF THE PREFERRED EMBODIMENTS

An agent used in the method for recovery of photo-inhibited immunologic ability of the present invention is, for example, a crude active fraction obtained by adding ammonium sulfate to an extract obtained from Gracilaria sp., using an aqueous salt solution, to a final concentration of 20% to 40% saturated solution to conduct a first stage of salting-out, so as to eliminate the precipitated contaminants, and further adding ammonium sulfate to the extract to a final concentration of 60% to 80% saturated solution to conduct a second stage of salting-out, so as to recover a crude active fraction as a precipitate, and then dissolving the precipitate in a suitable solution, followed by a simultaneous dialysis-isoelectric precipitation treatment; or a heat-treated crude active fraction obtained by conducting a heat treatment at 100° C. for 1 to 10 minutes before and after the aforementioned simultaneous dialysis-isoelectric precipitation treatment, so as to eliminate contaminant proteins; or a chromatographically purified active fraction obtained by separating components by chromatography and then collecting a fraction that exhibits activity of activating cellular immunocompetence, and in particular, activity of enhancing autoimmunity and recovering the photo-inhibited immunocompetence of cells with photo-inhibited immunity; or a liquid substance obtained by fractionating fractions with a molecular weight of 100,000 or more by gel filtration chromatography. The aforementioned dialysis is carried out using a dialysis membrane such as regenerated cellulose tube.

The aforementioned saturated concentration of ammonium sulfate is determined in accordance with the “table showing the relationship between the additive amount of crystalline ammonium sulfate and the concentration (% saturation)” described in Green, A. A. & Hughes, W. L., (1955), Methods in Enzymology, vol. 1, pp. 67-90.”

Examples of an aqueous salt solution used in the method of the present invention may include: a physiological saline; a phosphate buffer solution; a Tris-hydrochloric acid buffer solution; and a liquid obtained by adding to these solutions at least one selected from among sodium chloride, potassium chloride, zinc sulfate, zinc chloride, 2-mercaptoethanol, and dithiothreitol. Of these, a phosphate buffer solution, a Tris-hydrochloric acid buffer solution, and a liquid obtained by adding to these solutions at least one selected from among sodium chloride, potassium chloride, zinc sulfate, and 2-mercaptoethanol, are particularly preferable.

The above obtained liquid substance comprises hemagglutinin containing sugar as a main component. Sugar, wherein the rate of galactose contained in monosaccharide that constitutes the sugar is 70% to 100%, and particularly 90% to 100%, is preferably used herein. When the aforementioned liquid substance is used as the agent for recovery of photo-inhibited immunocompetence of the present invention, it may comprise a protein as well as sugar, wherein the mass of the protein to that of the sugar is 0.4 or less.

It is to be noted that the sugar is assayed by the phenol sulfuric acid method using galactose as a standard sample, and that the protein is assayed by the Lowry method using bovine serum albumin as a standard sample.

As a raw material for the agent used in the method of the present invention, Gracilaria sp. is used. In particular, Gracilaria verrucosa, Gracilaria chorda, and subspecies thereof are preferable. Among others, Gracilaria sp., which is characterized in that neither male gametophytes nor female gametophytes are detectable as mature bodies in the nature and only tetrasporophytes are detectable as mature bodies, and which grows in a natural seawater area, into which fresh water is mixed, is more preferable; and an immature unialgal culture strain produced from Gracilaria sp., which is characterized in that neither male gametophytes nor female gametophytes are detectable as mature bodies in the nature and only tetrasporophytes are detectable as mature bodies, and which grows in a natural seawater area, into which fresh water is mixed, or an algal body by proliferation of the immature unialgal culture strain is most preferable. A preferred place to collect such Gracilaria sp., characterized in that neither male gametophytes nor female gametophytes are detectable as mature bodies in the nature and only tetrasporophytes are detectable as mature bodies may be a natural seawater area, into which fresh water is mixed, is preferable. Among others, the Gracilaria sp. collected in and around the middle of the Katsuura River in the estuary of the Katsuura River, Tokushima city, Tokushima prefecture, Japan, is more preferable. In the present invention, Gracilaria sp. includes: (1) marine algae belonging to Gracilaria sp.; (2) marine algae belonging to Gracilariopsis sp.; and (3) marine algae that have been previously classified into Gracilariopsis sp.

Examples of Gracilaria sp. of Japanese origin may include marine algae classified into Gracilariales, Gracilariaceae, in accordance with the Non-Patent Document “Shin Nihon Kaiso-shi Nihonsan Kaisorui Soran (Marine algae of Japan, Overview of Marine algae of Japanese origin), Tadao Yoshida, Uchida Rokakuho Pub., 1998.”

These red algae exist in cold sea, but the majority thereof particularly exist in warm sea. The red algae distribute over almost all seaboard regions in Japan, and they are used as expanders for agar or garnishings served with sliced raw fish.

In a preferred production method of the aforementioned agent, (A) a step of extracting a water-soluble fraction, (B) a step of separating a crude active fraction, and (C) a step of purifying a component for activating cellular immunocompetence, as necessary, are carried out successively on the aforementioned red alga raw material.

Each of the aforementioned steps will be described more in detail. First, in step (A), an aqueous salt solution such as a physiological saline, a phosphate buffer solution, a Tris-hydrochloric acid buffer solution, or a liquid obtained by adding to these solutions at least one selected from among sodium chloride, potassium chloride, zinc sulfate, zinc chloride, 2-mercaptoethanol, and dithiothreitol, and preferably, a phosphate buffer solution, tris(hydroxymethyl)aminomethane-hydrochloric acid buffer solution (hereinafter referred to as Tris-HCl), or a liquid obtained by adding to these solutions at least one selected from among sodium chloride, potassium chloride, zinc sulfate, zinc chloride, and 2-mercaptoethanol, is added to red algae as raw materials. Thereafter, the obtained mixture is homogenized and then centrifuged, so as to obtain a crude extract that is a supernatant.

Subsequently, in step (B), ammonium sulfate is first added to the extract obtained in the above step (A) to a final concentration of 20% to 40% saturated solution to conduct a first stage of salting-out, so that the generated precipitate is eliminated by centrifugation. By this operation, contaminants such as pigment can be eliminated as precipitated fractions. Thereafter, ammonium sulfate is further added to the supernatant obtained by centrifugation to a final concentration of 60% to 80% saturated solution to conduct a second stage of salting-out, so that the generated precipitate is separated by centrifugation. Thereafter, the precipitated fraction is dissolved again in a buffer solution such as a phosphate buffer solution containing sodium chloride, and as desired, the solution is purified by dialysis to a buffer solution such as a phosphate buffer solution containing sodium chloride or the like, thereby obtaining a crude active fraction. This crude active fraction has high activity of recovering photo-inhibited immunocompetence, but its specific activity is not high. That is to say, contaminants still remain. Accordingly, it is not preferable to directly use this crude active fraction in the method for recovery of photo-inhibited immunocompetence of the present invention.

Subsequently, this crude active fraction is injected into a dialysis tube. Using a dialyzing fluid obtained by blowing carbon dioxide into distilled water or an aqueous solution with an appropriate pH value as a dialyzing fluid, a simultaneous dialysis-isoelectric precipitation treatment (hereinafter referred to as SDIP treatment) is carried out, so as to separate a precipitate from a soluble fraction, thereby recovering an SDIP-treated crude active fraction that is a precipitate with higher specific activity per unit mass than that of the above crude active fraction, or thereby separating an SDIP-treated crude active fraction that is a soluble fraction with higher specific activity per unit mass than that of the above soluble crude active fraction. Thereafter, the thus obtained fraction is captured.

The pH of the dialyzing fluid used herein may be determined, depending on the isoelectric point of a biopolymer, such as a protein, to be subjected to an isoelectric precipitation treatment. In order to prepare a dialyzing fluid with pH 5.5, (1) a method of blowing carbon dioxide gas into distilled water to adjust the pH, or (2) a method using a thin buffer solution with a concentration of approximately 4 mM, is applied. Since water distilled with a distilled water production device has pH of approximately pH 5.5, such distilled water can directly be used. In the present invention, the SDIP treatment is preferably carried out at 4° C.

An example of procedures for purifying the aforementioned crude active fraction precipitated by salting-out with ammonium sulfate will be described below. (1) The crude active fraction that is precipitated by salting-out with ammonium sulfate is dissolved in a minimum amount of buffer solution A [25 mM Tris-HCl (pH 7.6) containing 30 mM potassium chloride, 3 μM zinc sulfate, and 1 mM 2-mercaptoethanol]. (2) When the solution in which the crude active fraction has been dissolved again contains a high concentration of ammonium sulfate for the initial period of time, it is better not to use distilled water having no buffering action as a dialyzing fluid because pH is extremely changed due to it. Thus, 2.5 L of buffer solution A is used to eight dialysis tubes each containing approximately 50 ml of the aforementioned crude active fraction-redissolved solution, so as to initiate dialysis. The dialyzing fluid is exchanged with a fresh one twice a day. (3) Sulfate ions in the dialyzing fluid are analyzed by ion chromatography. After confirming that the concentration of ammonium sulfate has been decreased, the dialyzing fluid is exchanged with buffer solution B [a 10 mM sodium phosphate buffer solution (pH 7.0) containing 0.15 mM NaCl], and dialysis is carried out twice, and it is then exchanged with buffer solution C [a 10 mM sodium phosphate buffer solution (pH 7.0) containing 0.015 mM NaCl], and dialysis is carried out twice. Thereafter, the dialyzing fluid is exchanged with distilled water with pH 5.5.

Moreover, as desired, at the end of step (B), or before the SDIP treatment, a crude active fraction or SDIP-treated crude active fraction is subjected to a heat treatment at 100° C. for 1 to 10 minutes, and the precipitated contaminant proteins are then eliminated, so as to obtain a heat-treated crude active fraction. By the above described operations, a crude active fraction that had been subjected to both the SDIP treatment and the heat treatment was obtained. The SDIP-treated, heat-treated crude active fraction contains fewer contaminants than the crude active fraction obtained only by salting-out does, and it has high specific activity. Thus, the SDIP-treated, heat-treated crude active fraction can be directly used in the method for recovery of photo-inhibited immunocompetence of the present invention.

This crude active fraction obtained by subjecting to both an SDIP treatment and a heat treatment is further subjected to step (C), as desired. In step (C), the crude active fraction obtained in the aforementioned step (B) by subjecting to both an SDIP treatment and a heat treatment is further subjected to chromatography, as desired, so as to separate components. Thus, fractions having activity of activating cellular immunocompetence, and in particular, activity of recovering photo-inhibited immunocompetence, are captured, thereby obtaining a chromatographically purified active fraction. Examples of chromatography that is advantageously applied at this stage may include: ion exchange chromatography, gel filtration chromatography, hydrophobic interaction chromatography, and the combined use thereof.

Furthermore, a fraction having a molecular weight of 100,000 or more is fractionated by gel filtration chromatography, so as to obtain a purified fraction or purified sample of a liquid agent for recovery of photo-inhibited immunocompetence.

The term “a fraction having a molecular weight of 100,000 or more” is used herein to mean a fraction, which is obtained by gel filtration chromatography using a globular protein as a standard molecular weight substance, wherein the resultant value obtained by calculation of the molecular weight of an eluted fraction is found to be 100,000 or more.

In order to preferably produce an agent used in the method for recovery of photo-inhibited immunocompetence of the present invention, 0.1 ml of a purified sample of cellular immunocompetence-stimulating component having mitogenic ability is added to a TSK gel G3000 PWXL column, and thus it is subjected to gel filtration chromatography, so as to gather 0.1 ml each of eluted fraction from the gel filtration chromatography column. At this time, as standard molecular weight substances, thyroglobulin (molecular weight: 669,000), ferritin (molecular weight: 440,000), bovine serum albumin (molecular weight: 67,000), and ovalbumin (molecular weight: 43,000) are used. As a result, it is found that a peak having activity indicating a fraction, at which the cellular immunocompetence-stimulating component having mitogenic ability has been eluted, corresponds to a molecular weight of 5.64×10⁵.

The agent used in the method for recovery of photo-inhibited immunocompetence of the present invention is a novel agent derived from red algae. This agent is advantageous in that it is able to control agglutination activity due to ionic concentration, in that it has excellent recognition carbohydrate selectivity, in that it has activity of recovering photo-inhibited immunocompetence, such as activity of activating cellular immunocompetence, and in that it has carbohydrate binding ability even after being subjected to a heat treatment at 100° C. for 10 minutes.

This agent is useful as a material used for treatments or tests in clinical, medical, and biochemical fields, and also as an additive used in a cosmetic field.

The present invention will be described more in detail in the following examples. The examples are not intended to limit the scope of the invention.

### EXAMPLE 1

(A) Step of Extracting Water-Soluble Fraction

Gracilaria chorda [produced in the estuary of Yoshino River, Tokushima prefecture, Japan; hereinafter referred to as Gracilaria chorda (produced in the estuary region of Yoshino River)] was washed with an aqueous 0.15 M sodium chloride solution, and then subjected to sun drying, so as to obtain a dried product. Thereafter, 700 ml of a 100 mM phosphate buffer solution (pH 6.9) containing 0.15 M sodium chloride was added to 100 g of the above dried product, and the obtained mixture was homogenized. Thereafter, the homogenized solution was left at 4° C. for 6 hours, followed by centrifugation, so as to obtain a crude extract that was a supernatant.

(B) Step of Separating Crude Active Fraction

Subsequently, ammonium sulfate was added to the obtained crude extract to a final concentration of 35% saturated solution, so as to conduct a first stage of salting-out. After completion of the addition of ammonium sulfate, the mixture was left at 4° C. for 1 hour. Thereafter, the generated precipitate was eliminated by centrifugation. By this operation, contaminants such as pigment can be eliminated as precipitated fractions. Thereafter, ammonium sulfate was added to the supernatant obtained as a result of the centrifugation to a final concentration of 70% saturated solution. After completion of the addition of ammonium sulfate, the mixture was left at 4° C. overnight. Thereafter, the generated precipitate was fractionated by centrifugation. The thus fractionated precipitated fraction (a crude active fraction that was in a precipitated state) was dissolved in a minimum amount of buffer solution A [25 mM Tris-HCl (pH 7.6) containing 30 mM potassium chloride, 3 μM zinc sulfate, and 1 mM 2-mercaptoethanol], so as to obtain a liquid crude active fraction. Subsequently, an aliquot of the crude active fraction was dialyzed to a 100 mM phosphate buffer solution (pH 6.9) containing 0.15 M sodium chloride, and the hemagglutination activity on rabbit erythrocytes was measured. As a result, the activity was found to be 256 units, and the minimum protein concentration was found to be 0.438 μg/ml. Herein, the unit of hemagglutination activity was defined as the reciprocal of the maximum dilution rate of a sample whose hemagglutination activity could be detected. Specific activity and activity-recovery rate are shown in Table 14.

Subsequently, an SDIP treatment was carried out. That is to say, a liquid crude active fraction obtained by dissolving the precipitate of a crude active fraction obtained by salting-out with ammonium sulfate in a minimum amount of the aforementioned buffer solution A, was placed into a dialysis tube, and a simultaneous dialysis-isoelectric precipitation treatment was performed thereon, while the dialyzing fluid was exchanged with a fresh one at suitable intervals of time. 2.5 L of buffer solution A was used to eight dialysis tubes each containing approximately 50 ml of the aforementioned crude active fraction-redissolved solution, so as to initiate dialysis. The dialyzing fluid is exchanged with a fresh one twice a day. Sulfate ions in the dialyzing fluid were analyzed by ion chromatography. After confirming that the concentration of ammonium sulfate had been decreased, the dialyzing fluid was exchanged with buffer solution B [a 10 mM sodium phosphate buffer solution (pH 7.0) containing 0.15 mM NaCl], and dialysis was then carried out twice. Thereafter, it was exchanged with buffer solution C [a 10 mM sodium phosphate buffer solution (pH 7.0) containing 0.015 mM NaCl], and dialysis was then carried out twice. Thereafter, the dialyzing fluid was exchanged with distilled water (pH: approximately 5.5). As a dialysis tube, a film with a fractional molecular weight of 8,000 was used. This operation was carried out in a chamber with a low temperature of 4° C.

Three days after the exchange of the dialyzing fluid with distilled water (pH: approximately 5.5) (the dialyzing fluid was exchanged 6 times), the transparency of an internal dialyzing solution was reduced. After the dialysis was further continued for 1 day (the dialyzing fluid was further exchanged twice), it was confirmed that a precipitate was accumulated at the bottom of the dialysis tube. Thereafter, the dialysis was further carried out for 1 day (the dialyzing fluid was further exchanged twice). Thereafter, the upper and lower ends of the dialysis tube were held by hands, and it was moved up and down, so that the precipitate was suspended therein. The suspension was then centrifuged, so as to separate the precipitate from a soluble fraction. The precipitate was dissolved in a suitable solvent, so as to obtain a precipitate-redissolved fraction.

Thus, the precipitate-redissolved fraction and a soluble fraction could be obtained as SDIP-treated crude active fractions.

The aforementioned SDIP-treated crude active fraction was subjected to a heat treatment at 100° C. for 10 minutes, and contaminant proteins were eliminated by centrifugation, so as to obtain a crude active fraction that had been subjected to the heat treatment following the SDIP treatment.

(C) Step of Purifying Cellular Immunocompetence-Stimulating Component

Subsequently, the thus SDIP-treated, heat-treated crude active fraction was separated by ion exchange chromatography using TSK gel DEAE-5PW, and a fraction having a molecular weight of 100,000 or more was then fractionated by gel filtration chromatography, so as to obtain a purified sample. The minimum protein concentration in the obtained purified sample necessary for exhibiting hemagglutination activity on rabbit erythrocytes was found to be 0.438 μg/ml. These results show that hemagglutinin derived from red algae is effectively obtained, while it maintains its activity.

The purified sample was tested in terms of the ionic strength dependence of hemagglutination activity on rabbit erythrocytes. As a result, it was found that the hemagglutination activity was 4,096 units at a sodium chloride concentration of 0.15 M, and that it was 8 units at a sodium chloride concentration of 0.4 M.

After the purified sample had been subjected to a heat treatment at 100° C. for 10 minutes, the hemagglutination activity thereof was found to be 4,096 units. Thus, inactivation of the hemagglutination activity due to the heat treatment was not observed. Since the hemagglutination activity of hemagglutinin is considered to be an indicator of the sugar-binding activity thereof, it is found from the above results that the sugar-binding activity of the agent for recovery of photo-inhibited immunocompetence of the present invention is stable to heat.

In order to examine the mitogenic activity of the purified sample, a human lymphocyte blast transformation test was carried out. Such a human lymphocyte blast transformation test has been frequently used to measure the DNA synthetic ability of the peripheral blood lymphocytes of patients or healthy subjects and to compare them. It is considered that the results of this test involve general cell-mediated immune reaction ability. Examples of the measurement method may include: a method of counting the number of cells, in which chromosomes have appeared, in a fixed sample by stain; and a method involving morphological observation. In the present example, however, the method of measuring incorporation of ³H-thymidine into a cell nucleus was applied. Lymphocytes were collected from the analytes of three healthy subjects, and were then subjected to an experiment.

As a culture solution, an aqueous solution was prepared by dissolving 1.05 g of RPMI 1640, 0.2 g of NaHCO₃, 10,000 units of penicillin, 10 mg of streptomycin, and 10 ml of fetal bovine serum, in 100 ml of pure water. The solution was sterilized by filtration through a filter. Thereafter, it was placed in a small bottle depending on the amount used, and it was then hermetically sealed. It was conserved at −20° C.

As a control mitogen, red kidney bean lectin was dissolved in a culture solution, and it was adjusted to a concentration of 10 to 50 μg/ml. The solution was dispensed in small sterilized test tubes, and they were hermetically sealed, followed by conservation at −20° C.

Lymphocytes were separated as follows. That is, lymphocytes were separated from heparinized blood by the Ficoll-Conray method, and they were then washed with CMF-PBS (pH 7.0) 3 times. The separated lymphocytes were suspended in 1 ml of the culture solution, and the number thereof was then counted. Subsequently, the number of lymphocytes was adjusted to 5×10⁵ cells/ml by addition of the culture solution, so as to obtain a lymphocyte suspension.

The lymphocytes were cultured as follows. That is, 200 μl of the lymphocyte suspension was dispensed into each well of a microplate. Thereafter, 20 μl each of a purified sample acting as a mitogen solution, a control mitogen acting as a positive control, and a phosphate buffer solution (PBS) acting as a negative control, were dispensed into each well. Thereafter, the mixture was cultured in the air of 5% CO₂ at 37° C. in a wet state for 3 days. Eight hours before completion of the culture, ³H-thymidine was dispensed to each well, so that the final concentration thereof in the culture solution became 1 μCi/ml.

The activity was measured as follows. That is, while the inside of the well was harvested with a salt solution using Labo-MASH, the cells were gathered on a glass fiber filter. These cells on the filter were subjected to continuous suction, so as to wash them (approximately 20 seconds; approximately 1.5 ml of a physiological saline). Subsequently, a cell-immobilized portion was removed from the glass filter, and it was then placed in a counting vial. After the portion had been sufficiently dried, 5 ml of a toluene scintillator (0.1 g of POPO+5 g of PPO/L toluene) acting as a liquid scintillator was dispensed to each vial, using a dispenser, and the activity was measured using a scintillation counter. Measurement was carried out 3 times for each analyte, and a mean value was obtained. The results are shown in Table 1.

TABLE 1

| | | | | | Comparison |
|---|---|---|---|---|---|
| | | | | | with |
| | Incorporated amount of ³H-thymidine (cpm) | | | | negative |
| | | | | Mean | control |
| | Analyte 1 | Analyte 2 | Analyte 3 | value | (times) |
| Purified | 58038 | 60120 | 53663 | 57274 | 166.0 |
| sample | | | | | |
| Positive | 38128 | 42006 | 35228 | 38454 | 111.5 |
| control | | | | | |
| Negative | 299 | 397 | 338 | 345 | 1.0 |
| control | | | | | |

This table shows that the agent for recovery of photo-inhibited immunocompetence of the present invention exhibits higher mitogenic activity than those of the previously known hemagglutinins derived from land plants.

### EXAMPLE 2

(A) Step of extracting water-soluble fraction

500 g (wet mass) of Gracilaria chorda (produced in the estuary of Yoshino River) was washed with an aqueous 0.15 M sodium chloride solution, and then freeze-dried at −30° C. Thereafter, a 0.5 M tris(hydroxymethyl)aminomethane-hydrochloric acid buffer solution (pH 8.2) containing 30 mM potassium chloride, 3 1 M zinc sulfate, and 5 mM 2-mercaptoethanol was used as an extraction buffer solution, and 800 ml of such an extraction buffer solution was added to finely crushed freeze-dried marine alga (corresponding to 500 g (wet mass) of Gracilaria chorda). The obtained mixture was homogenized, and the thus homogenized solution was left at 4° C. for 6 hours, followed by centrifugation, so as to obtain a crude extract that was a supernatant.

(B) Step of Separating Crude Active Fraction

Subsequently, this crude extract was fractionated by the same procedures as described in (B) step of separating crude active fraction of Example 1, so as to obtain a liquid crude active fraction (referred to as fraction I). Thereafter, an aliquot of the crude active fraction was dialyzed to a 100 mM phosphate buffer solution (pH 6.9) containing 0.15 M sodium chloride, and the hemagglutination activity on rabbit erythrocytes was measured. As a result, the activity was found to be 256 units, and the minimum protein concentration was found to be 0.438 μg/ml.

Another aliquot of fraction I was subjected to a heat treatment at a temperature of 100° C. for 10 minutes, separately, and contaminant proteins were then eliminated by centrifugation, so as to obtain a crude active fraction (referred to as fraction II).

The residual fraction I was subjected to an SDIP treatment in the same manner as in step (B) of Example 1, so as to obtain a crude active fraction (referred to as fraction III).

Fraction III was subjected to a heat treatment at a temperature of 100° C. for 10 minutes, and contaminant proteins were then eliminated by centrifugation, so as to obtain a crude active fraction that had been subjected to a heat treatment after an SDIP treatment (referred to as fraction IV).

(C) Step of Purifying Cellular Immunocompetence-Stimulating Component

The hemagglutination activity of the thus obtained purified sample on rabbit erythrocytes was found to be 4,096 units. These results show that the agent for recovery of photo-inhibited immunocompetence of the present invention is obtained, while it maintains its activity.

The purified sample was tested in terms of the ionic strength dependence of hemagglutination activity on rabbit erythrocytes. As a result, it was found that the hemagglutination activity was 4,096 units at a sodium chloride concentration of 0.15 M, and that it was 8 units at a sodium chloride concentration of 0.4 M.

After the purified sample had been subjected to a heat treatment at 100° C. for 10 minutes, the hemagglutination activity thereof was found to be 4,096 units. Thus, inactivation of the hemagglutination activity due to the heat treatment was not observed. Since the hemagglutination activity of hemagglutinin is considered to be an indicator of the sugar-binding activity thereof, it is found from the above results that the sugar-binding activity of the agent for recovery of photo-inhibited immunocompetence of the present invention is stable to heat.

A human lymphocyte blast transformation test was then carried out utilizing incorporation of ³H-thymidine. Fractions I, II, III and IV and the purified sample were measured in terms of mitogenic activity. In this case, all materials necessary for cell culture, a microplate, a cell harvester, a glass fiber filter, a counting vial, ³H-thymidine, a toluene scintillator (0.1 g of POPO+5 g of PPO/L toluene), and a liquid scintillation counter were prepared in an aseptic manner. In addition, all operations using these materials were also carried out in an aseptic manner.

As a culture solution, an aqueous solution was prepared by dissolving 1.05 g of RPMI 1640, 0.2 g of NaHCO₃, 10,000 units of penicillin, 10 mg of streptomycin, and 10 ml of fetal bovine serum, in 100 ml of pure water. The solution was sterilized by filtration through a filter. Thereafter, it was placed in a small bottle depending on the amount used, and it was then hermetically sealed. It was conserved at −20° C. The freeze-dried product could be conserved and used for 2 months in this state. When used, it was used up, in order not to repeat freezing and thawing.

Lymphocytes were separated from heparinized blood by the Ficoll-Conray method. Thereafter, they were washed with CMF-PBS (pH 7.0) 3 times, and then suspended in 1 ml of a culture solution. The number of lymphocytes was then counted. Subsequently, the number thereof was adjusted to 5×10⁵ cells/ml by addition of a culture solution.

200 μl of the lymphocyte suspension was dispensed into each well of a microplate, and the lymphocytes were cultured therein.

Subsequently, the microplate containing lymphocytes was placed in a clean booth. The experiment was carried out using three experiment groups. A control experiment group that had been left in a clean booth for 30 minutes without irradiation of ultraviolet rays was used as experiment group A. An experiment group wherein ultraviolet rays was applied from above to lymphocytes in a microplate for 30 minutes was used as experiment group B. An experiment group wherein ultraviolet rays was applied from above to lymphocytes in a microplate for 16 hours was used as experiment group C. Ultraviolet rays was applied as follows. A microplate was placed in a chromatography viewing portable darkroom (manufactured by Funakoshi), and long-wavelength ultraviolet rays (365 nm) was applied from 6 W handy-type UV lamp UVL-56 black rays lamp (manufactured by Funakoshi) attached to the upper portion of the dark room. The strength of the ultraviolet rays at 365 nm was measured using a MODEL UVX-36 sensor (manufactured by Funakoshi) connected with a digital-type UVX RADIOMETER ultraviolet meter (manufactured by Funakoshi). The strength of the ultraviolet rays was measured to be 0.63 mW/cm² at the position of the microplate.

Subsequently, for each experiment group, as mitogen solutions, fractions I, II, III and IV, a purified sample, and a phosphate buffer solution (PBS) were dispersed to each well, at an amount of 20 μl each. A diluted solution obtained by diluting each fraction or the purified sample with a buffer solution (10 to 320 times diluted) was prepared, and it was subjected to the experiment. The amount (cpm) of ³H-thymidine incorporated into each fraction or the purified sample was obtained by multiplying the measurement value of the diluted solution by a dilution magnification, so as to convert the value to that of a stock solution. Subsequently, the above obtained mixture was cultured in the air of 5% CO₂ at 37° C. in a wet state for 3 days. Eight hours before completion of the culture, ³H-thymidine was dispensed to each well, so that the final concentration thereof in the culture solution became 1 μCi/ml.

The activity was measured as follows. That is, while the inside of the well was harvested with a salt solution using Labo-MASH, the cells were gathered on a glass fiber filter. These cells on the filter were subjected to continuous suction, so as to wash them (approximately 20 seconds; approximately 1.5 ml of a physiological saline). Subsequently, a cell-immobilized portion was removed from the glass filter, and it was then placed in a counting vial. After the portion had been sufficiently dried, 5 ml of a liquid scintillator was dispensed to each vial, using a dispenser, and the activity was measured using a scintillation counter. The experiment was carried out using lymphocytes from the analytes of 3 subjects who differed from the 3 healthy subjects used in Example 1 (hereinafter referred to as analyte a, analyte b, and analyte c). The experiment was repeated 3 times under certain experimental conditions. The mean value of 3 times of measurements was adopted, and such mean values for the analytes are shown in the following tables. The results of analyte a are shown in Table 2, the results of analyte b are shown in Table 3, and the results of analyte c are shown in Table 4. With regard to the specific activity of each of fractions I, II, III, and IV, the results of analyte a are shown in Table 5, the results of analyte b are shown in Table 6, and the results of analyte c are shown in Table 7.

In Tables 2 to 7, the activity means activity of recovering photo-inhibited immunocompetence. The activity is represented by a value obtained by subtracting the amount (cpm) of ³H-thymidine incorporated into lymphocytes, to which 20 μl of the negative control had been added, from the amount (cpm) of ³H-thymidine incorporated into lymphocytes, to which 20 μl of the fraction had been added. The experiment group shown in Tables 5 to 7 is experiment group B.

TABLE 2

| | Incorporated amount | | |
|---|---|---|---|
| | of ³H-thymidine (cpm) | | |
| | Experiment | Experiment | Experiment |
| | group A | group B | group C |
| Fraction I | 104067 | 102080 | 67783 |
| Fraction II | 112227 | 110010 | 71063 |
| Fraction III | 103813 | 101540 | 66083 |
| Fraction IV | 102830 | 101010 | 65787 |
| Purified sample | 641083 | 607693 | 422050 |
| Negative control | 171 | 128 | 89 |

TABLE 3

| | Incorporated amount | | |
|---|---|---|---|
| | of ³H-thymidine (cpm) | | |
| | Experiment | Experiment | Experiment |
| | group A | group B | group C |
| Fraction I | 126693 | 122650 | 82833 |
| Fraction II | 149083 | 138100 | 90120 |
| Fraction III | 133047 | 129120 | 84407 |
| Fraction IV | 130280 | 127800 | 83823 |
| Purified sample | 819507 | 773027 | 508933 |
| Negative control | 209 | 148 | 103 |

TABLE 4

| | Incorporated amount | | |
|---|---|---|---|
| | of ³H-thymidine (cpm) | | |
| | Experiment | Experiment | Experiment |
| | group A | group B | group C |
| Fraction I | 164613 | 159640 | 108667 |
| Fraction II | 179233 | 172900 | 108220 |
| Fraction III | 162793 | 159180 | 104023 |
| Fraction IV | 157027 | 154200 | 100537 |
| Purified sample | 1023333 | 970543 | 675053 |
| Negative control | 260 | 190 | 124 |

TABLE 5

| | | | Total | Rate of | | | Rate of | |
|---|---|---|---|---|---|---|---|---|
| | | | mass | recovery | | | recovery | |
| | | | of | of | | Total | of | Specific |
| | Protein | Volume | protein | protein | Activity | activity | activity | activity |
| Fraction | (mg/ml) | (ml) | (mg) | (%) | (cpm) | (cpm) | (%) | (cpm/mg) |
| I | 0.495 | 100 | 49.50 | 100.0 | 1.0208 × 10⁵ | 5.1040 × 10⁸ | 100.0 | 1.031 × 10⁷ |
| II | 0.355 | 80 | 28.40 | 57.4 | 1.1001 × 10⁵ | 4.4004 × 10⁸ | 86.2 | 1.549 × 10⁷ |
| III | 0.132 | 105 | 13.86 | 28.0 | 1.0154 × 10⁵ | 5.3309 × 10⁸ | 104.4 | 3.846 × 10⁷ |
| IV | 0.063 | 84 | 5.29 | 10.7 | 1.0101 × 10⁵ | 4.2424 × 10⁸ | 83.1 | 8.020 × 10⁷ |

TABLE 6

| | | | | | | | Rate | |
|---|---|---|---|---|---|---|---|---|
| | | | Total | Rate of | | | of | |
| | | | mass | recovery | | | recovery | |
| | | | of | of | | Total | of | Specific |
| | Protein | Volume | protein | protein | Activity | activity | activity | activity |
| Fraction | (mg/ml) | (ml) | (mg) | (%) | (cpm) | (cpm) | (%) | (cpm/mg) |
| I | 0.495 | 100 | 49.50 | 100.0 | 1.2265 × 10⁵ | 6.1325 × 10⁸ | 100.0 | 1.239 × 10⁷ |
| II | 0.355 | 80 | 28.40 | 57.4 | 1.3810 × 10⁵ | 5.5240 × 10⁸ | 90.1 | 1.945 × 10⁷ |
| III | 0.132 | 105 | 13.86 | 28.0 | 1.2912 × 10⁵ | 6.7788 × 10⁸ | 110.5 | 4.891 × 10⁷ |
| IV | 0.063 | 84 | 5.29 | 10.7 | 1.2780 × 10⁵ | 5.3676 × 10⁸ | 87.5 | 10.147 × 10⁷ |

TABLE 7

| | | | | Rate | | | Rate | |
|---|---|---|---|---|---|---|---|---|
| | | | Total | of | | | of | |
| | | | mass | recovery | | | recovery | |
| | | | of | of | | Total | of | Specific |
| | Protein | Volume | protein | protein | Activity | activity | activity | activity |
| Fraction | (mg/ml) | (ml) | (mg) | (%) | (cpm) | (cpm) | (%) | (cpm/mg) |
| I | 0.495 | 100 | 49.50 | 100.0 | 1.5964 × 10⁵ | 7.9820 × 10⁸ | 100.0 | 1.613 × 10⁷ |
| II | 0.355 | 80 | 28.40 | 57.4 | 1.7290 × 10⁵ | 6.9160 × 10⁸ | 86.6 | 2.435 × 10⁷ |
| III | 0.132 | 105 | 13.86 | 28.0 | 1.5918 × 10⁵ | 8.3570 × 10⁸ | 104.7 | 6.030 × 10⁷ |
| IV | 0.063 | 84 | 5.29 | 10.7 | 1.5420 × 10⁵ | 6.4764 × 10⁸ | 81.1 | 12.243 × 10⁷ |

As is clear from experiment group A shown in Tables 2 to 4, the amount of ³H-thymidine incorporated into the agent for recovery of photo-inhibited immunocompetence of the present invention containing the crude active fraction obtained in Example 2 and that of the above agent containing the purified sample are significantly larger than that of the negative control (600 times or more and 3,700 times or more, respectively). Thus, it is found that these agents exhibit excellent mitogenic activity.

Moreover, as is clear from the mean values of the negative control shown in Tables 2 to 4, it is found that the amount of ³H-thymidine incorporated into cells, namely, immunity, is decreased by irradiation of ultraviolet ray. Furthermore, as is clear from the results of experiment groups B and C, it is found that incorporation of ³H-thymidine into cells is promoted by addition of the agent for recovery of photo-inhibited immunocompetence of the present invention, even if ultraviolet rays has been applied to the cells.

From the aforementioned results, it is found that addition of the crude active fraction or purified sample thereof of the agent for recovery of photo-inhibited immune function to human lymphocytes, whose immunity such as DNA synthetic ability (incorporation of ³H-thymidine or the like) has been decreased by a ultraviolet rays irradiation treatment, enables an increase in the immunity of the above human lymphocytes, such as DNA synthetic ability. In addition, if such an ultraviolet rays irradiation time is within 30 minutes, the DNA synthetic ability can be increased to the same level as the case of adding the agent for recovery of photo-inhibited immune function to human lymphocytes to which no ultraviolet rays has been applied. Even if an ultraviolet rays has been applied to human lymphocytes for 16 hours, the DNA synthetic ability thereof can be increased to 50% or more of the case of adding the agent for recovery of photo-inhibited immune function to human lymphocytes to which no ultraviolet rays has been applied. It is found that the amount of ³H-thymidine incorporated in the case of the agent for recovery of photo-inhibited immune function of the present invention is much larger than that of the negative control, to which no ultraviolet rays has been applied.

### EXAMPLE 3

An agent for recovery of photo-inhibited immunocompetence was produced in the same manner as in Example 2 with the exception that 500 g (wet mass) of an immature unialgal culture strain derived from Gracilaria chorda [produced in the estuary of Katsuura River, Tokushima city, Tokushima prefecture, Japan; hereinafter referred to as Gracilaria chorda (Katuura River)] was used as a raw material, instead of 500 g (wet mass) of Gracilaria chorda (produced in the estuary of Yoshino River). The Gracilaria chorda (Katuura River) belongs to Gracilaria sp., which is characterized in that neither male gametophytes nor female gametophytes are detectable as mature bodies in the nature, but only tetrasporophytes are detectable as mature bodies, and which grows in a natural seawater area, into which fresh water is mixed.

Separation and purification were carried out in the same manner as in Example 2 with the exception that the immature unialgal culture strain derived from Gracilaria chorda (Katsuura River) was used as a raw material, so as to obtain a crude active fraction (referred to as fraction V). Thereafter, fraction V was treated by heating, so as to obtain a crude active fraction (referred to as fraction VI), and fraction V was treated by SDIP, so as to obtain a crude active fraction (referred to as fraction VII). Moreover, fraction VII was treated by heating, so as to obtain a crude active fraction (referred to as fraction VIII). Thereafter, with regard to the thus obtained fractions V, VI, VII and VIII, a purified sample thereof, and a phosphate buffer solution (PBS) used as a negative control, the activity of the agent for recovery of photo-inhibited immunocompetence was measured in the same manner as in Example 2 with the exception that lymphocytes used were collected from the analytes of thee subjects who differed from those in Example 2 (hereinafter referred to as analytes d, e, and f).

The experiment was repeated 3 times under certain experimental conditions. The mean value of 3 times of measurements was adopted. The results of analyte d are shown in Table 8, the results of analyte e are shown in Table 9, and the results of analyte f are shown in Table 10. With regard to the specific activity of each of fractions V, VI, VII, and VIII, the results of analyte d are shown in Table 11, the results of analyte e are shown in Table 12, and the results of analyte f are shown in Table 13.

In Tables 8 to 13, the activity means activity of recovering photo-inhibited immunocompetence. The activity is represented by a value obtained by subtracting the amount (cpm) of ³H-thymidine incorporated into lymphocytes, to which 20 μl of the negative control had been added, from the amount (cpm) of ³H-thymidine incorporated into lymphocytes, to which 20 μl of the fraction had been added. The experiment group shown in Tables 11 to 13 is experiment group B.

TABLE 8

| | Incorporated amount | | |
|---|---|---|---|
| | of ³H-thymidine (cpm) | | |
| | Experiment | Experiment | Experiment |
| | group A | group B | group C |
| Fraction V | 187003 | 183120 | 121600 |
| Fraction VI | 203073 | 198700 | 129900 |
| Fraction VII | 193007 | 189440 | 123027 |
| Fraction VIII | 189607 | 186230 | 122293 |
| Purified sample | 1188230 | 1136673 | 789007 |
| Negative control | 176 | 132 | 92 |

TABLE 9

| | Incorporated amount | | |
|---|---|---|---|
| | of ³H-thymidine (cpm) | | |
| | Experiment | Experiment | Experiment |
| | group A | group B | group C |
| Fraction V | 220023 | 213210 | 144063 |
| Fraction VI | 261023 | 241110 | 158063 |
| Fraction VII | 228407 | 221610 | 144880 |
| Fraction VIII | 224590 | 220210 | 145067 |
| Purified sample | 1466037 | 1380280 | 908623 |
| Negative control | 221 | 157 | 109 |

TABLE 10

| | Incorporated amount | | |
|---|---|---|---|
| | of ³H-thymidine (cpm) | | |
| | Experiment | Experiment | Experiment |
| | group A | group B | group C |
| Fraction V | 260493 | 252180 | 171893 |
| Fraction VI | 290770 | 280340 | 178067 |
| Fraction VII | 269277 | 262960 | 179947 |
| Fraction VIII | 266523 | 260210 | 169963 |
| Purified sample | 1700237 | 1603387 | 1123007 |
| Negative control | 275 | 201 | 131 |

TABLE 11

| | | | | Rate | | | | |
|---|---|---|---|---|---|---|---|---|
| | | | Total | of | | | Rate of | |
| | | | mass | recovery | | | recovery | |
| | | | of | of | | Total | of | Specific |
| | Protein | Volume | protein | protein | Activity | activity | activity | activity |
| Fraction | (mg/ml) | (ml) | (mg) | (%) | (cpm) | (cpm) | (%) | (cpm/mg) |
| V | 0.440 | 105 | 46.20 | 100.0 | 1.8312 × 10⁵ | 9.6138 × 10⁸ | 100.0 | 2.081 × 10⁷ |
| VI | 0.318 | 58 | 27.03 | 58.5 | 1.9870 × 10⁵ | 8.4448 × 10⁸ | 87.8 | 3.1242 × 10⁷ |
| VII | 0.122 | 110 | 13.42 | 29.0 | 1.8944 × 10⁵ | 10.419 × 10⁸ | 108.4 | 7.764 × 10⁷ |
| VIII | 0.059 | 90 | 5.31 | 11.5 | 1.8623 × 10⁵ | 8.3804 × 10⁸ | 87.2 | 15.782 × 10⁷ |

TABLE 12

| | | | | | | | Rate | |
|---|---|---|---|---|---|---|---|---|
| | | | Total | Rate of | | | of | |
| | | | mass | recovery | | | recovery | |
| | | | of | of | | Total | of | Specific |
| | Protein | Volume | protein | protein | Activity | activity | activity | activity |
| Fraction | (mg/ml) | (ml) | (mg) | (%) | (cpm) | (cpm) | (%) | (cpm/mg) |
| V | 0.440 | 105 | 46.20 | 100.0 | 2.1321 × 10⁵ | 11.194 × 10⁸ | 100.0 | 2.4228 × 10⁷ |
| VI | 0.318 | 85 | 27.03 | 58.5 | 2.4110 × 10⁵ | 10.247 × 10⁸ | 91.5 | 3.7910 × 10⁷ |
| VII | 0.122 | 110 | 13.42 | 29.0 | 2.2161 × 10⁵ | 12.189 × 10⁸ | 108.9 | 9.0827 × 10⁷ |
| VIII | 0.059 | 90 | 5.31 | 11.5 | 2.2021 × 10⁵ | 9.909 × 10⁸ | 88.5 | 18.661 × 10⁷ |

TABLE 13

| | | | Total | Rate of | | | Rate of | |
|---|---|---|---|---|---|---|---|---|
| | | | mass | recovery | | | recovery | |
| | | | of | of | | Total | of | Specific |
| | Protein | Volume | protein | protein | Activity | activity | activity | activity |
| Fraction | (mg/ml) | (ml) | (mg) | (%) | (cpm) | (cpm) | (%) | (cpm/mg) |
| V | 0.440 | 105 | 46.20 | 100.0 | 2.5218 × 10⁵ | 13.239 × 10⁸ | 100.0 | 2.8656 × 10⁷ |
| VI | 0.318 | 85 | 27.03 | 58.5 | 2.8034 × 10⁵ | 11.914 × 10⁸ | 90.0 | 4.4077 × 10⁷ |
| VII | 0.122 | 110 | 13.42 | 29.0 | 2.6296 × 10⁵ | 14.463 × 10⁸ | 109.2 | 10.777 × 10⁷ |
| VIII | 0.059 | 90 | 5.31 | 11.5 | 2.6021 × 10⁵ | 11.709 × 10⁸ | 88.4 | 22.051 × 10⁷ |

### COMPARATIVE EXAMPLE 1

A crude active fraction was obtained in the same manner as in Example 1 with the exception that a separation treatment with 50% by mass of ethanol was carried out [refer to Phytochemistry, vol. 27, pp. 2,063-2,067 (1988)], instead of the separation treatment by 2 stages of salting-out using ammonium sulfate in the step of separating a crude active fraction of Example 1-(B). The hemagglutination activity, specific activity, and activity-recovery rate of this crude active fraction on rabbit erythrocytes are shown in Table 14. For comparison, the results of Example 1 are also shown in the table.

### COMPARATIVE EXAMPLE 2

In accordance with a common method [the method described in Comp. Biochem. Physiol., vol. 102B, pp. 445-449 (1992)], hemagglutinin derived from red algae was obtained. The hemagglutination activity, specific activity, and activity-recovery rate of the obtained crude active fraction are shown in Table 14. The minimum protein concentration, which indicates the hemagglutination activity of a purified sample on rabbit erythrocytes, was found to be 32.6 μg/ml. This value corresponded to approximately one seventieth of the specific activity in Example 1.

TABLE 14

| | Crude active fraction | | |
|---|---|---|---|
| | Hemagglutination | | Activity- |
| | activity¹⁾ | Specific activity | recovery rate²⁾ |
| | (unit) | (unit/mg protein) | (%) |
| Example1 | 256 | 3372.9 | 62.4 |
| Comparative | 4 | 53.4 | 5.0 |
| Example 1 | | | |
| Comparative | 16 | 149.5 | 19.5 |
| Example 2 | | | |

Note
¹⁾The hemagglutination activity was obtained by successively diluting the crude active fraction and calculating from the maximum dilution rate indicating the hemagglutination activity.
Note
²⁾The activity-recovery rate was calculated, defining the hemagglutination activity of the total amount of a crude extract solution as 100%.

### COMPARATIVE EXAMPLE 3

With regard to hemagglutinin having a molecular weight of 50,000 purified by the same method as that in Comparative example 2, the ionic concentration dependence of the hemagglutination activity on rabbit erythrocytes was analyzed. The hemagglutination activity at a concentration of 0.15 M sodium chloride and that at a concentration of 0.4 M sodium chloride are shown in Table 15. The hemagglutination activity was not dependent on ionic concentration. For comparison, the results of Example 1 are also shown in the table.

### COMPARATIVE EXAMPLE 4

25 mg of Con A (manufactured by Wako Pure Chemical Industries, Co., Ltd.) was dissolved in 100 ml of a phosphate buffer solution, and the ionic concentration dependence of the hemagglutination activity on rabbit erythrocytes was analyzed. The hemagglutination activity at a concentration of 0.15 M sodium chloride and that at a concentration of 0.4 M sodium chloride are shown in Table 15. The hemagglutination activity was not dependent on ionic concentration.

TABLE 15

Hemagglutination activity
on rabbit erythrocytes
(unit)¹⁾

| | 0.15M NaCl | 0.4M NaCl |
|---|---|---|
| Example 1 | 4096 | 8 |
| Comparative | 1024 | 1024 |
| Example 3 | | |
| Comparative | 64 | 64 |
| Example 4 | | |

Note
¹⁾The hemagglutination activity was obtained by successively diluting the purified hemagglutinin and calculating from the maximum dilution rate indicating the hemagglutination activity.

After Con A had been subjected to a heat treatment at 100° C. for 10 minutes, no hemagglutination activity was detected. Thus, it was found that the hemagglutination activity was inactivated by a heat treatment.

As is clear from Table 14, all of the hemagglutination activity, specific activity, and activity-recovery rate of the agent for recovery of photo-inhibited immunocompetence of the present invention consisting of the crude active fraction obtained in Example 1 are higher than those of Comparative examples 1 and 2. The activity-recovery rate thereof is approximately 12 times higher than that of Comparative example 1, and approximately 3 times higher than that of Comparative example 2. The specific activity thereof is approximately 63 times higher than that of Comparative example 1, and approximately 23 times higher than that of Comparative example 2. Moreover, from Table 15, it is found that the purified hemagglutinin of Example 1 differs from those of Comparative examples 3 and 4, and that the hemagglutination activity on rabbit erythrocytes is controlled by ionic concentration.

What is claimed is:

1. A method for recovery of photo-inhibited immunological activity, comprising administration to a human body a liquid extract having physiological activity that is obtained from Gracilaria sp., using an aqueous salt solution.

2. The method according to claim 1, wherein the Gracilaria sp. is Gracilaria verrucosa, Gracilaria chorda, or subspecies thereof.

3. The method according to claim 1, wherein the Gracilaria sp. is characterized in that neither male gametophytes nor female gametophytes are detectable as mature bodies in the nature and only tetrasporophytes are detectable as mature bodies, and wherein the Gracilaria sp. grows in a natural seawater area, into which fresh water is mixed.

4. The method according to claim 1, wherein the Gracilaria sp. is an immature unialgal culture strain derived from Gracilaria sp., which is characterized in that neither male gametophytes nor female gametophytes are detectable as mature bodies in the nature and only tetrasporophytes are detectable as mature bodies, and which grows in a natural seawater area, into which fresh water is mixed, or an algal body by proliferation of the immature unialgal culture strain.

5. The method according to claim 1, characterized in that the liquid extract has a property to agglutinate sheep erythrocytes treated with pronase, and in that the agglutination activity is not inhibited by monosaccharide or disaccharide, but is inhibited by fetuin or asialofetuin.

6. The method according to claim 1, characterized in that the agglutination activity of the liquid extract on rabbit erythrocytes is changed according to ionic strength.

7. The method according to claim 1, characterized in that the physiological activity of the liquid extract is activity of activating cellular immunocompetence.

8. The method according to claim 1, characterized in that the physiological activity of the liquid extract is blast transformation activity on human lymphocytes.

9. The method according to claim 1, characterized in that the liquid extract promotes incorporation of tritium-labeled thymidine into a cell nucleus.

10. The method according to claim 1, characterized in that the liquid extract does not lose carbohydrate binding activity by a heat treatment at 100° C. for 10 minutes.

11. The method according to claim 1, wherein the liquid extract comprises sugar and protein, and the content of said protein to that of the sugar is 0.4 or less (mass ratio).

12. The method according to claim 1, wherein the liquid extract comprises 1% to 60% by mass of sulfuric acid.

13. The method according to claim 1, characterized in that the liquid extract comprises a component eluted in a fraction that corresponds to a molecular weight of 100,000 or more in gel filtration chromatography using a globular protein as a standard molecular weight substance.

14. A method for producing a liquid extract having physiological activity, comprising the steps of (a) obtaining an extract from Gracilaria sp., using an aqueous salt solution; (b) adding ammonium sulfate to the obtained extract to a final concentration of 20% to 40% saturated solution to conduct a first stage of salting-out, so as to eliminate the precipitated contaminants; (c) further adding ammonium sulfate to the extract to a final concentration of 60% to 80% saturated solution to conduct a second stage of salting-out, so as to recover a crude active fraction exhibiting physiological activity as a precipitate; and (d) dissolving the recovered precipitate in a solvent, so as to prepare a solution.

15. The production method according to claim 14, comprising the steps of (a) obtaining an extract from Gracilaria sp., using an aqueous salt solution; (b) adding ammonium sulfate to the obtained extract to a final concentration of 20% to 40% saturated solution to conduct a first stage of salting-out, so as to eliminate the precipitated contaminants; (c) further adding ammonium sulfate to the extract to a final concentration of 60% to 80% saturated solution to conduct a second stage of salting-out, so as to recover a crude active fraction exhibiting physiological activity as a precipitate; (d) dissolving the recovered precipitate in a solvent, so as to prepare a solution; and (e) subjecting said solution to a heat treatment at a temperature of 100° C. for 1 to 10 minutes, so as to precipitate and eliminate contaminants.

16. The production method according to claim 15, wherein the aqueous salt solution is a phosphate buffer solution containing sodium chloride.

17. The production method according to claim 14, wherein the aqueous salt solution is a tris(hydroxymethyl)aminomethane-hydrochloric acid buffer solution containing at least one selected from among potassium chloride, zinc sulfate, and 2-mercaptoethanol.

18. A method for producing an extract having physiological activity, comprising the steps of (a1) obtaining an extract from Gracilaria sp., using an aqueous salt solution; (b1) adding ammonium sulfate to the obtained extract to a final concentration of 20% to 40% saturated solution to conduct a first stage of salting-out, so as to eliminate the precipitated contaminants; (c1) further adding ammonium sulfate to the extract to a final concentration of 60% to 80% saturated solution to conduct a second stage of salting-out, so as to separate and eliminate the generated precipitate; (d1) dissolving said precipitate in a buffer solution, so as to prepare a solution containing a substance exhibiting physiological activity and impurities; and (e1) allowing this solution to come into contact with a dialyzing fluid that has been adjusted to the isoelectric point of the substance exhibiting physiological activity, via a dialysis membrane, so as to transfer low molecular weight impurities to the dialyzing fluid and eliminate them, and at the same time, precipitating a biologically active polymer from a solution containing high molecular weight impurities and recovering it.

19. The production method according to claim 18, comprising a step of subjecting the solution containing a substance exhibiting physiological activity and impurities to a heat treatment at a temperature of 100° C. for 1 to 10 minutes before allowing the solution to come into contact with the dialysis membrane, so as to eliminate contaminant proteins from the solution in advance.

20. The production method according to claim 18, comprising a step of purifying the extract by gel filtration chromatography, so as to capture fractions with a molecular weight of 100,000 or more.

21. The production method according to claim 18, wherein the aqueous salt solution is a phosphate buffer solution containing sodium chloride.

22. The production method according to claim 18, wherein the aqueous salt solution is a tris(hydroxymethyl)aminomethane-hydrochloric acid buffer solution containing at least one selected from among potassium chloride, zinc sulfate, and 2-mercaptoethanol.

23. The production method according to claim 18, wherein the dialyzing fluid is a distilled water or buffer solution, the pH of which has been adjusted with carbon dioxide.

24. The production method according to claim 18, wherein the dialysis membrane is a tube made from a regenerated cellulose film.

25. A method for producing an extract having physiological activity, comprising the steps of (a2) obtaining an extract from Gracilaria sp., using an aqueous salt solution; (b2) adding ammonium sulfate to the obtained extract to a final concentration of 20% to 40% saturated solution to conduct a first stage of salting-out, so as to eliminate the precipitated contaminants; (c2) further adding ammonium sulfate to the extract to a final concentration of 60% to 80% saturated solution to conduct a second stage of salting-out, so as to separate the generated precipitate; (d2) dissolving the extract in a buffer solution, so as to prepare a solution containing a substance exhibiting physiological activity and impurities; and (e2) allowing this solution to come into contact with a dialyzing fluid that has been adjusted to the isoelectric point of high molecular weight impurities, via a dialysis membrane, so as to transfer low molecular weight impurities to the dialyzing fluid and eliminate them, and at the same time, precipitating and separating the high molecular weight impurities, so as to obtain a solution exhibiting physiological activity.

26. The production method according to claim 25, comprising a step of subjecting the solution containing a substance exhibiting physiological activity and impurities to a heat treatment at a temperature of 100° C. for 1 to 10 minutes before allowing the solution to come into contact with the dialysis membrane, so as to eliminate contaminant proteins from the solution in advance.

27. The production method according to claim 25, comprising a step of purifying the extract by gel filtration chromatography, so as to capture fractions with a molecular weight of 100,000 or more.

28. The production method according to claim 25, wherein the aqueous salt solution is a phosphate buffer solution containing sodium chloride.

29. The production method according to claim 25, wherein the aqueous salt solution is a tris(hydroxymethyl)aminomethane-hydrochloric acid buffer solution containing at least one selected from among potassium chloride, zinc sulfate, and 2-mercaptoethanol.

30. The production method according to claim 25, wherein the dialyzing fluid is a distilled water or buffer solution, the pH of which has been adjusted with carbon dioxide.

31. The production method according to claim 26, wherein the dialysis membrane is a tube made from a regenerated cellulose film.
